(19) United States
(12) Patent Application Publication
(10) Pub. No.: US 2009/0018174 A1
(43) Pub. Date: Jan. 15, 2009

(54) MACROLIDES AS ANTI-INFLAMMATORY AGENTS

(76) Inventors: Pradip Kumar Bhatnagar, Exton, PA (US); Anjan Chakrabarti, Gurgaon (IN); Biswajit Das, Gurgaon (IN); Malini Bajpai, Lucknow (IN); Abhijit Ray, New Delphi (IN)

Correspondence Address: RANBAXY INC., 600 COLLEGE ROAD EAST, SUITE 2100, PRINCETON, NJ, 08540, US

(21) Appl. No.: 12/093,041

(22) PCT Filed: Nov. 8, 2006
(86) PCT No.: PCT/IB06/54171

§ 371 Date: Sep. 18, 2008

(30) Foreign Application Priority Data
Nov. 8, 2005 (IN) 2965/DEL/2005

(51) Int. Cl. A61K 31/365; A61K 31/424

(52) U.S. Cl. 514/375; 514/450

(57) ABSTRACT

Provided herein are macrolide derivatives that can be useful in treating or preventing inflammatory diseases. Also provided are pharmaceutical compositions comprising one or more macrolide derivatives optionally together with one or more pharmaceutically acceptable carriers, excipients, diluents or mixtures thereof, as well as methods for treating or preventing inflammatory diseases.

### FIELD OF THE INVENTION

Provided herein are macrolide derivatives that can be useful in treating or preventing inflammatory diseases. Also provided are pharmaceutical compositions comprising one or more macrolide derivatives optionally together with one or more pharmaceutically acceptable carriers, excipients, diluents or mixtures thereof, as well as methods for treating or preventing inflammatory diseases.

### BACKGROUND OF THE INVENTION

Macrolides are known to be effective bacteriostatic and bactericidal agents, used for the treatment of range of bacterial infections. They are effective against gram positive and some gram-negative bacteria, also particularly against atypical pneumonia organisms.

Besides, several studies have proved that macrolides are significantly effective in alleviating inflammatory responses and are known to be useful in mitigation of asthma since three decades (Itkins, I. H. Journal of Allergy, 45, 146-62 (1970)). Erythromycin A derivatives have been shown to possess in vitro anti-inflammatory properties (M. T. Labro, J. Antimicrob. Chemother., 41 (suppl B), 37-46, (1998)). Macrolides, particularly 14-membered ring macrolides, such as erythromycin and clarithromycin, have been shown to reduce the production of endothelin-1, a bronchoconstrictor, in a fashion similar to that of corticosteroids. Roxithromycin was found to be more potent than azithromycin and clarithromycin, while erythromycin showed the least anti-inflammatory activity (D. Wales, Thorax, 54 (Suppl 2), 558-562, (1999)). The potency of roxithromycin is owed to the better cell penetration.

Inflammation is the first response of the immune system to infection or irritation. The inflammatory response begins with a release of inflammatory chemicals into the extracellular fluid. Various leukocytes are involved in the initiation and maintenance of inflammation. Neutrophils, eosinophils and monocytes are phagocytic in nature. Along with basophils they secrete leukotrienes, prostaglandins, histamine and other chemicals that promote inflammation. The leukocytes are further stimulated to maintain inflammation through lymphocytes: T cells, B cells, and antibodies. Mast cells, also release potent inflammatory mediators, such as histamine proteases, chemotactic factors, cytokines and metabolites of arachidonic acid, in response to activation of stretch receptors; macrophages release TNF-α, IL-1 in response to activation of toll-like receptors. Increased permeability of blood vessels, increased blood flow, migration and accumulation of white blood cells are factors that contribute to the formation of inflammatory exudate, which accumulates in the interstitial spaces in the area of injury and results in heat, swelling, redness and pain. Roxithromycin has been shown to interfere with the production and/or release of inflammatory substances, which can be as active as some non-steroidal anti-inflammatory agents (e.g., nimesulide and indomethacin). Patients with diffuse panbronchiolitis have shown distinct improvement after treatment with macrolides. The 5 year survival rate of 55% has increased to 94% survival rate of 10 years.

Macrolides can be effectively used in the prophylaxis and/or treatment of respiratory diseases viz. bronchitis, bronchiectasis, rhinitis, cystic fibrosis, emphysema, asthma, pneumonia, sinusitis, tonsillitis, chronic obstructive pulmonary disease or adult respiratory distress syndrome; renal diseases viz. glomerulonephritis (with and without nephritic syndrome); autoimmune diseases viz., rheumatoid arthritis, gout, autoimmune thyroiditis, autoimmune bullous dermatoses, multiple sclerosis or systemic lupus erythematosus; skin diseases viz., psoriasis, eczema, allergic dermatitis, neurodermatitis, or pruritis; cardiovascular diseases viz., myocardial ischemia, congestive heart failure or atherosclerosis; inflammatory bowel disease viz., Crohn's disease, ulcerative colitis, or colitis, others which include, Reiter's syndrome, leukocytoclastic vasculitis, conjunctivitis, uveoretinitis, septic shock, rheumatoid spondylitis, osteoarthritis, septic arthritis or inflammation associated with organ transplant rejection. Inflammation associated with smoking and other environmental factors is also encompassed.

PCT Publication No. WO 05/030786 discloses 3′-N-substituted-3-O-substituted erythronolide A derivatives as antibacterial agents. PCT Application No. PCT/US2005/027875 discloses ketolide derivatives as antibacterial agents. PCT Publication Nos. WO 06/035301, WO 06/013409 and WO 06/046112 disclose macrolides as antibacterial agents. U.S. Pat. No. 6,455,576 discloses 3′-des-dimethylamino-9-oxyimino macrolides having anti-inflammatory activity. PCT Publication No. WO 02/087596 discloses novel therapeutic indication of azithromycin for treatment of non-infective inflammatory diseases. PCT Publication No. WO 04/005310 discloses new compounds, compositions and methods for treatment of inflammatory diseases and conditions. PCT Publication No. WO 04/005309 discloses novel non-steroidal anti-inflammatory substances, compositions and methods for their use. Antiinflammatory effects of macrolide have been described in the literature. For example, F. Scaglione in, J. Antimicrob. Chemother. 41, Suppl. B, 47-50 (1998) describes the comparative anti-inflammatory effects of roxithromycin, azithromycin and clarithromycin. J. Med. Chem. 47, 4950-4957, (2004) describes the anti-inflammatory activity of macrolide MLD987 for inflammatory lung diseases.

The existing macrolides are highly effective antibacterial agents, which makes it difficult to use them in the treatment of inflammatory diseases. Thus, there remains a need for novel macrolides as anti-inflammatory agents, devoid of antibacterial activity.

### SUMMARY OF THE INVENTION

Accordingly, provided are macrolide derivatives for treating or preventing inflammatory diseases or disorders, wherein the macrolide derivatives are compounds having the structure of Formula I,

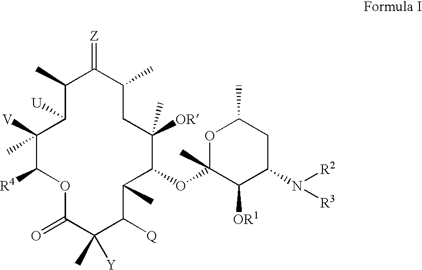

pharmaceutically acceptable salts, pharmaceutically acceptable solvates, polymorphs, prodrugs, stereoisomers, tautomeric forms, N-oxides and metabolites thereof, wherein:
Y can be hydrogen, halogen, cyano or alkyl;
Q can be oxo, thio, —O—(C=T)R⁵, wherein
   T can be oxygen or sulfur;
   R⁵ can be alkyl, alkoxy, aryl or heteroaryl;
R¹ can be hydrogen or a hydroxy protecting group;
R² and R³ can be independently alkyl, alkenyl, alkynyl, cycloalkyl, aryl, heterocycle, aralkyl, (heterocycle)alkyl or COR⁶, wherein
   R⁶ can be hydrogen, alkyl, aryl or aralkyl, with the provisio that R² and R³ simultaneously are not methyl;
R′ can be hydrogen, alkyl or —(CH₂)_{q}—R⁷—, wherein
   q is an integer from 1 to 4; and
   R⁷ can be no atom (i.e., a bond), alkenyl or alkynyl;
Z can be O, S, NOR⁶ (wherein R⁶ is the same as defined earlier);
U and V can be independently hydrogen, hydroxy, OR¹, NH(CH₂)R⁸, wherein
   R¹ is the same as defined earlier;
   R⁸ can be aryl or heteroaryl; or
   U and V when taken together can also form a ring of Formula A or Formula B,

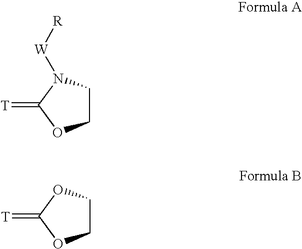

wherein
   W can be alkenyl, -G(CH₂)ₘJ-, —CR⁹R¹⁰, —NR⁹— or —SO₂;
      G can be no atom, —CO, —CS or —SO₂;
      m can be an integer of from 0 to 6;
      R⁹ and R¹⁰ can be independently hydrogen or alkyl;
      J can be no atom, —CR⁹R¹⁰ or N(R¹²)(CH₂)ₘ;
         R¹² can be hydrogen, alkyl, alkylene, alkynyl, COR¹¹ or —(CH₂)ₘ—R¹¹, wherein
         R¹¹ can be alkyl, aryl or heterocycle; and
         m, R⁹ and R¹⁰ are the same as defined earlier;
   R can be hydrogen, aryl or heterocycle;
   T is as defined earlier; and
R⁴ can be alkyl, alkenyl or alkynyl.

Such compounds may include one or more of the following embodiments. For example in one embodiment, provided herein are macrolide derivatives having the structure of Formula Ia,

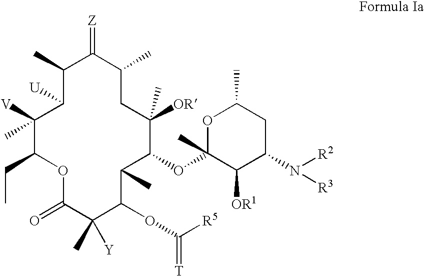

wherein Y is hydrogen and R¹, R², R³, R′, Z, U, V, T and R⁵ are the same as defined for the compound of Formula I above.

In another embodiment, provided herein are macrolide derivatives of Formula Ia,

wherein Y is halogen, R¹, R², R³, R′, Z, U, V, T and R⁵ are the same as defined for the compound of Formula I.

In another embodiment, provided herein are macrolide derivatives having the structure of Formula 1b,

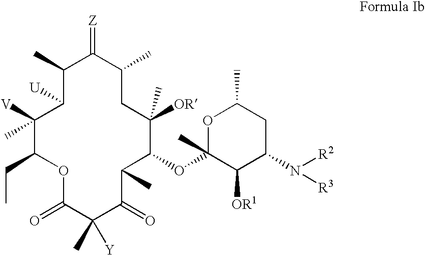

wherein Y is hydrogen, and R¹, R², R³, R′, Z, U and V are the same as defined for the compound of Formula I above.

In another embodiment, provided herein are macrolide derivatives of Formula 1b, wherein Y is halogen and R¹, R², R³, R′, Z, U, V, T and R⁵ are the same as defined for the compound of Formula I.

In yet another embodiment, provided herein are macrolide derivatives having the structure of Formula 2,

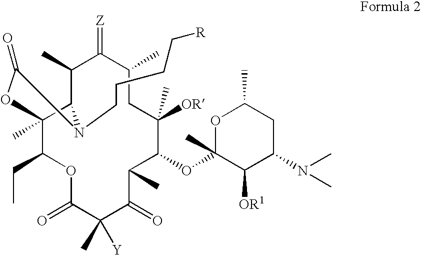

and pharmaceutically acceptable salts, pharmaceutically acceptable solvates, polymorphs, prodrugs, stereoisomers, tautomeric forms, N-oxides or metabolites thereof, wherein Y can be hydrogen or methyl R¹, R′, and Z are the same as defined for Formula I and R can be aryl or heterocycle.

Also provided herein are compounds for treating or preventing inflammatory diseases, including for example:
3-O-(2-Fluorophenyl)acetyl-5-O-[3′-N-desmethyl-3′-N-(4-fluoro)benzyl]desosaminyl-6-O-methyl erythronolide A,
3-O-(3-Fluorophenyl)acetyl-5-O-(3′-N-desmethyl-3′-N-cyclopropyl)desosaminyl-6-O-methyl erythronolide A,
3-O-(4-Fluorophenyl)acetyl-5-O-[3′-N-desmethyl-3′-N-(3-hydroxy)benzyl]desosaminyl-6-O-methyl erythronolide A,
3-O-(4-Fluorophenyl)acetyl-5-O-(3′-N-desmethyl-3′-N-propargyl)desosaminyl-6-O-methyl erythronolide A,
3-O-(3-Fluorophenyl)acetyl-5-O-(3′-N-desmethyl-3′-N-allyl)desosaminyl-6-O-methyl erythronolide A,
3-O-(4-Fluorophenyl)acetyl-5-O-(3′-N-desmethyl-3′-N-allyl)desosaminyl-6-O-methyl erythronolide A,
3-O-(2-Fluorophenyl)acetyl-5-O-(3′-N-desmethyl-3′-N-propargyl)desosaminyl-6-O-methyl erythronolide A,
3-O-(3-Fluorophenyl)acetyl-5-O-(3′-N-desmethyl-3′-N-propargyl)desosaminyl-6-O-methyl erythronolide A,
3-O-(2-Fluorophenyl)acetyl-5-O-(3′-N-desmethyl-3′-N-allyl)desosaminyl-6-O-methyl erythronolide A,
3-O-(4-Fluorophenyl)acetyl-5-O-(3′-N-desmethyl-3′-N-(4-nitro) benzyl)desosaminyl-6-O-methyl erythronolide A,
3-O-(4-Nitrophenyl)acetyl-5-O-(3′-N-desmethyl-3′-N-ethyl)desoaminyl-6-O-methyl erythronolide A,
3-O-(2-Pyridyl)acetyl-5-O-(3′-N-desmethyl-3′-N-cyclopropylmethyl)desosaminyl-6-O-methyl erythronolide A,
3-O-(2-Naphthyl)acetyl-5-O-(3′-N-desmethyl-3′-N-benzyl)desosaminyl-6-O-methyl erythronolide A,
3-O-(2,4-Difluorophenyl)acetyl-5-O-(3′-N-desmethyl-3′-N-benzyl) desosaminyl-6-O-methyl erythronolide A,
2-ax-Fluoro-5-O-(3′-N-desmethyl-3′-N-ethyl)-11,12-dideoxy-3-O-decladinosyl-6-O-methyl-3-oxo-12,11-[oxycarbonyl-((4-(pyrrolo-(2,3-b)-pyridin-1-yl)-butyl)-imino)]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(3H)-imidazol-[4,5-b]pyridin-3-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(1H)-imidazol-[4,5-b]pyridin-1-yl)-butyl)-imino]-erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-pyrrolo-[2,3-b]pyridin-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-indol-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-quinolin-4-ylmethyl)-2-aminoethyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N-pyridine-4-ylmethyl)-2-aminoethyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-nitro)-indol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((1H)-imidazo[4,5-c]pyridin-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-2-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((3H)-imidazo-[4,5-c]pyridin-3-yl)-butyl)-imino)]erythromycin A,
2α-Fluoro-5-O-(3′-N-desmethyl-3′-N-ethyl)-1,12-dideoxy-3-O-decladinosyl-6-O-methyl-3-oxo-12,11-[oxycarbonyl-((4-(pyridin-4-yl)-butyl)-imino)]erythromycin A
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-3-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-(benzotriazol-1-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-(benzoimidazol-1-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-((3H)-imidazo-[4,5-b]pyridin-3-yl)-butyl)-imino)]erythromycin A,
2α-Fluoro-5-O-(3′-N-desmethyl-3′-N-isopropyl)-1,12-dideoxy-3-O-decladinosyl-6-O-methyl-3-oxo-12,11-[oxycarbonyl-((4-((4-phenyl)-imidazol-1-yl)-butyl)-imino)]erythromycin A,
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-(benzotriazol-2-yl)-butyl)-imino)]erythromycin A,
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-(indol-1-yl)-butyl)-imino)]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-2-ylmethyl)-2-aminoethyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-quinolin-4-ylmethyl)-2-aminoethyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-4-ylmethyl)-2-aminoethyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-quinoline-4-ylmethyl)-2-aminoethyl)-imino]erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyridin-3-yl-methyl-benzimidazol-1-yl)-butyl)-imino)]erythromycin A,
5-O-(3′-N-desmethyl-3″-N-ethyl)desosaminyl-11,12-dideoxy-3-O-decladinosyl-6-O-methyl-3-oxo-12,11-[oxycarbonyl-((4-(4-pyridin-3-yl-imidazol-1-yl)-butyl)-imino)]erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(imidazo[4,5-b]pyridin-1-yl)-butyl)-imino)]erythromycin A,
2α-Fluoro-5-O-(3′-N-desmethyl-3′-N-isopropyl)-1,12-dideoxy-3-O-decladinosyl-6-O-methyl-3-oxo-12,11-[oxycarbonyl-((4-((5-nitro)-indol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-fluoro)-indol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-2-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A,
2α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-pyridin-3-yl)-imidazol-1-yl)-butyl)-imino]erythromycin A,
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A,
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-(benzotriazol-1-yl)-butyl)-imino)]erythromycin A,
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-pyridin-3-yl)-imidazol-1-yl)-butyl)-imino)]erythromycin A,
2α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((N′-acetyl-N′-pyridine-3-ylmethyl)-2-aminoethyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(3H)imidazo[4,5-b]pyridin-3-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-phenyl)-imidazol-1-yl)-butyl)-imino]erythromycin A,
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-(3H)imidazo[4,5-b]pyridin-3-yl)-butyl)-imino]erythromycin A,
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(quinolin-8-yl)-butyl)-imino)]erythromycin A,
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A,
2α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-pyridin-3-yl)-imidazo)-1-yl)-butyl)-imino)]erythromycin A,
11,12-dideoxy-3-O-decladinosyl-3-O-(2-pyridylacetyl)-5-O-(3′-N-desmethyl-3′-N-propargyl)-6-O-methyl-12,11-[oxycarbonyl-((4-benzoimidazo-1-yl)-butyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A,
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyridin-2-yl-1H-imidazol-1-yl)-butyl)-imino))]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl-pyrazol-1-yl)-butyl)-imino))erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((3-pyrrolo[2,3-b]pyridin-1-yl)-propyl)-hydrazo)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((indazol-2-yl)-butyl)-imino))]erythromycin A
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-amino-9H-purin-9-yl butylimino)]erythromycin A,
11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(isoquinolin-5-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-allyl-3-(9-(4-amino-butyl)-9H-purin-6-yl)-urea)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-2-yl-1H-imidazol-1-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(1H-imidazol[4,5-c]pyridin-1-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-3-yl)-1H-imidazol-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-purin-9-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-purin-9-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-isoquinolin-5-yl)-propyl)-hydrazo)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(isoquinolin-5-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-imidazol-1-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-3-yl-1H-imidazol-1-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-furan-3-yl-1H-imidazol-1-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-2-yl-1H-imidazol-1-yl)-propyl)-hydrazo)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol-1-yl)propyl)-hydrazo)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-furan-2-yl-1H-imidazol-1-yl)-butyl)-imino))]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-thiophen-2-yl-1H-imidazol-1-yl)-butyl)-imino))]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-2-yl)-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-4-([1,4′]-bipyrazol-1′-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-3-yl)-propyl)-hydrazo)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-thiophen-3-yl-phenoxy)-propyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(3-thiophen-3-yl-phenoxy)-propyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-thiazolyl)-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiazol-2-yl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(1H-imidazol[4,5-b]pyridin-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-tetrazol-1-yl)-imidazol-1-yl)-butylimino)]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-Imidazo[4,5-b]pyridine-2-yl)-butyl)-imino)erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(1H-benzimidazol-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Tetrazol-1-yl-imidazol-1-yl)-butyl)-imino)erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-Benzoimidazol-2-yl)-butyl)-imino)erythromycin A,
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(4-imidazol[4,5-b]pyridin-1-yl)-butylimino)]erythromycin A,
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(6-amino-9H-purin-9-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[4-(pyrazol-1-yl)-imidazol-1-yl)butylimino]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-([1,4′]-biimidazol-1′-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([1,4′]-biimidazol-1′-yl)butylimino)]erythromycin A,
11,12-dideoxy-3-O-decladinosyl-3-O-(2-methylpropanoyl)-5-O-(3′-N-desmethyl-3′-N-allyl)-6-O-methyl-12,11-[oxycarbonyl-(4-(imidazo[4,5-b]pyridin-3-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyrazol-1-yl)-imidazol-1-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiazol-2-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(4-pyrazol-1-yl)-imidazol-1-yl)-propyl)-hydrazo]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-(1H-imidazo[4,5-b]pyridine-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(oxazol-5-yl)-imidazol-1-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[3,3′]-bithiophenyl-5-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-3-yl)-tetrazol-2-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(6-chloropyridin-3-yl)-imidazol-1-yl]butylimino)]erythromycin A,
11,12-dideoxy-3-O-decladinosyl-3-O-(2-methylpropanoyl)-5-O-(3′-N-desmethyl-3′-N-allyl)-6-O-methyl-12,11-[oxycarbonyl-4-(4-(4-(furan-2-yl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-([1,4′]-bipyrazol-1-yl)-)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(2-methoxy-phenyl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-methoxy-phenyl)-1H-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-furan-2-yl-thiophen-2-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-furan-2-yl-thiophen-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A,
11,12-dideoxy-3-O-decladinosyl-3-O-(2-nitrophenyl acetyl)-5-O-(3′-N-desmethyl-3′-N-allyl)-6-O-methyl-12,11-[oxycarbonyl-(4-benzimidazol-1-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(4-pyrazol-1-yl]-imidazol-1-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(3-aminophenyl)-imidazol-1-yl)-butylimino)]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Thiophen-3-yl-pyrazol-1-yl)-butyl)-imino)erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Thiophen-2-yl-pyridin-3-yl)-butyl)-imino)-erythromycin A, or
   pharmaceutically acceptable salts, pharmaceutically acceptable solvates, polymorphs, prodrugs, stereoisomers, tautomeric forms, N-oxides and metabolites thereof.

Also provided herein are methods for treating or preventing inflammatory diseases or disorders selected from respiratory diseases, renal diseases, autoimmune diseases, skin diseases, cardiovascular diseases and other diseases which include, Reiter's syndrome, leukocytoclastic vasculitis, conjunctivitis, uveoretinitis, septic shock, atherosclerosis, Crohn's disease, ulcerative colitis, colitis, rheumatoid spondylitis, osteoarthritis or septic arthritis.

Also provided herein are pharmaceutical compositions comprising therapeutically effective amounts of one or more compounds of Formula I or pharmaceutically acceptable salts, pharmaceutically acceptable solvates, stereoisomers, polymorphs, N-oxide, prodrugs or metabolites thereof, together with one or more pharmaceutically acceptable carriers, excipients, diluents or mixtures thereof. Such compositions can be used to treat or prevent inflammatory diseases or disorders.

Such pharmaceutical compositions may include one or more of the following embodiments. For example in one embodiment, pharmaceutical compositions comprise therapeutically effective amounts of one or more compounds of Formula Ia or pharmaceutically acceptable salts, pharmaceutically acceptable solvates, stereoisomers, polymorphs, N-oxide, prodrugs or metabolites thereof, together with one or more pharmaceutically acceptable carriers, excipients, diluents or mixtures thereof.

In another embodiment, pharmaceutical compositions comprise therapeutically effective amounts of one or more compounds of Formula 1b or pharmaceutically acceptable salts, pharmaceutically acceptable solvates, stereoisomers, polymorphs, N-oxide, prodrugs or metabolites thereof, together with one or more pharmaceutically acceptable carriers, excipients, diluents or mixture thereof.

In another embodiment, pharmaceutical compositions comprise therapeutically effective amounts of one or more compounds of Formula 2 or pharmaceutically acceptable salts, pharmaceutically acceptable solvates, stereoisomers, polymorphs, N-oxide, prodrugs or metabolites thereof, together with one or more pharmaceutically acceptable carriers, excipients, diluents or mixture thereof.

Also provided are combinations of one or more compounds of Formula I with other active agents in the same or different class or that can be used in therapy of inflammatory diseases to achieve additive or synergistic effects. Such agents may include one or more of steroidal anti-inflammatory agents, non-steroidal anti-inflammatory agents, COX-2 inhibitors, macrolide immunosuppresives, p-38 MAP Kinase inhibitors, local anesthetics, beta-2 agonists, 5-lipooxygenase inhibitors, phosphodiesterase inhibitors, MMP inhibitors, TNF-alpha inhibitors, caspase inhibitors, VLA4 antagonists, PAF antagonists or mixtures thereof.

Also provided herein are combination pharmaceutical compositions for treating or preventing inflammatory diseases or disorders.

Other aspect and properties of this matter will be set forth in description which follows, and will be apparent from the description or may be learnt by the practice thereof. However, it should be understood that the following detailed description is given by way of illustration only, since various changes and modifications within the scope of the invention will become apparent to those skilled in the art and are intended to encompass within the scope herein.

### DETAILED DESCRIPTION OF THE INVENTION

In accordance with one aspect, provided are macrolide derivatives for treating or preventing the inflammatory diseases or disorders, wherein macrolide derivatives are compounds having the structure of Formula I,

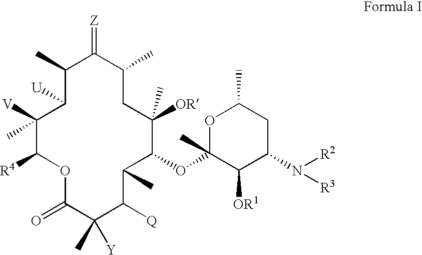

or pharmaceutically acceptable salts, pharmaceutically acceptable solvates, polymorphs, prodrugs, stereoisomers, tautomeric forms, N-oxides and metabolites thereof, wherein
Y can be hydrogen, halogen, cyano or alkyl;
Q can be oxo, thio, —O—(C=T)R⁵, wherein
   T can be oxygen or sulfur;
   R⁵ can be alkyl, alkoxy, aryl or heteroaryl;
R¹ can be hydrogen or a hydroxy protecting group;
R² and R³ can be independently alkyl, alkenyl, alkynyl, cycloalkyl, aryl, heterocycle, aralkyl, (heterocycle)alkyl or COR⁶, wherein
   R⁶ can be hydrogen, alkyl, aryl or aralkyl, with the provisio that R² and R³ simultaneously are not methyl;
R′ can be hydrogen, alkyl or —(CH₂)_{q}—R⁷—, wherein
   q is an integer from 1 to 4; and
   R⁷ can be no atom (i.e., a bond), alkenyl or alkynyl;
Z can be O, S, NOR⁶ (wherein R⁶ is the same as defined earlier);
U and V can be independently hydrogen, hydroxy, OR¹, NH(CH₂)R⁸, wherein
   R¹ is the same as defined earlier;
   R⁸ can be aryl or heteroaryl; or
   U and V when taken together can also form a ring of Formula A or Formula B,

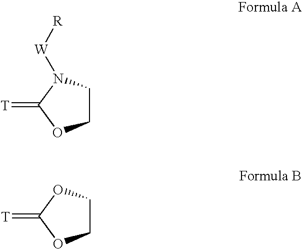

wherein
   W can be alkenyl, -G(CH₂)ₘJ-, —CR⁹R¹⁰, —NR⁹— or —SO₂;
      G can be no atom, —CO, —CS or —SO₂;
      m can be an integer of from 0 to 6;
      R⁹ and R¹⁰ can be independently hydrogen or alkyl;
      J can be no atom, —CR⁹R¹⁰ or N(R¹²)(CH₂)ₘ;
         R¹² can be hydrogen, alkyl, alkylene, alkynyl, COR¹¹ or —(CH₂)ₘ—R¹¹, wherein
         R¹¹ can be alkyl, aryl or heterocycle; and
         m, R⁹ and R¹⁰ are the same as defined earlier;
   R can be hydrogen, aryl or heterocycle;
   T is as defined earlier; and
R⁴ can be alkyl, alkenyl or alkynyl.

Such compounds can be prepared by, for example, methods described in WO05/030786 (U.S. application Ser. No. 10/573,275), WO06/080954 (U.S. application Ser. No. ______), WO06/013409 (U.S. application Ser. No. ______), WO06/035301 (U.S. application Ser. No. ______) and WO06/046112 (U.S. application Ser. No. ______), each of which are incorporated herein by reference.

In one embodiment, provided herein are macrolide derivatives having the structure of Formula Ia,

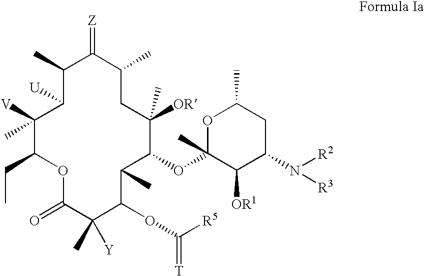

wherein Y is hydrogen and R¹, R², R³, R′, Z, U, V, T and R⁵ are the same as defined for the compound of Formula I.

In another embodiment, provided herein are macrolide derivatives of Formula Ia, wherein Y is halogen and R¹, R², R³, R′, Z, U, V, T and R⁵ are the same as defined for the compound of Formula I.

In another embodiment, provided herein macrolide derivatives having the structure of Formula 1b,

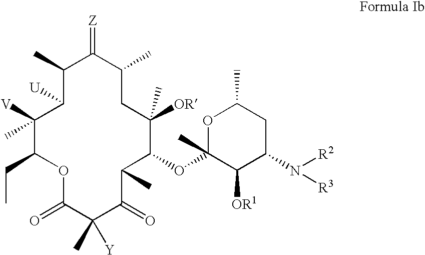

wherein Y is hydrogen and R¹, R², R³, R′, Z, U and V are the same as defined for the compound of Formula I.

In another embodiment, provided herein macrolide derivatives of Formula Ib, wherein Y is halogen and R¹, R², R³, R′, Z, U, V, T and R⁵ are the same as defined for the compound of Formula I.

In yet another embodiment, provided herein are macrolide derivatives having the structure of Formula 2,

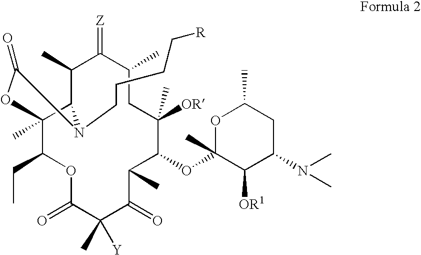

or pharmaceutically acceptable salts, pharmaceutically acceptable solvates, polymorphs, prodrugs, stereoisomers, tautomeric forms, N-oxides and metabolites thereof, wherein Y can be hydrogen or methyl; R can be aryl or heterocycle; and R¹, R′, and Z are the same as defined for the compound of Formula I.

Hydroxy protecting groups include those known to one of ordinary skill in the art. Hydroxy protecting also include, for example, acetyl, benzoyl, butyldiphenylsilyl, methylthiomethyl, or methoxy methyl.

Also provided herein are compounds for treating or preventing inflammatory diseases, including for example:
3-O-(2-Fluorophenyl)acetyl-5-O-[3′-N-desmethyl-3′-N-(4-fluoro)benzyl]desosaminyl-6-O-methyl erythronolide A (Compound No. 1),
3-O-(3-Fluorophenyl)acetyl-5-O-[3′-N-desmethyl-3′-N-cyclopropyl)desosaminyl-6-O-methyl erythronolide A (Compound No. 2),
3-O-(4-Fluorophenyl)acetyl-5-O-[3′-N-desmethyl-3′-N-(3-hydroxy)benzyl]desosaminyl-6-O-methyl erythronolide A (Compound No. 3),
3-O-(4-Fluorophenyl)acetyl-5-O-(3′-N-desmethyl-3′-N-propargyl)desosaminyl-6-O-methyl erythronolide A (Compound No. 4),
3-O-(3-Fluorophenyl)acetyl-5-O-(3′-N-desmethyl-3′-N-allyl)desosaminyl-6-O-methyl erythronolide A (Compound No. 5),
3-O-(4-Fluorophenyl)acetyl-5-O-(3′-N-desmethyl-3′-N-allyl)desosaminyl-6-O-methyl erythronolide A (Compound No. 6),
3-O-(2-Fluorophenyl)acetyl-5-O-(3′-N-desmethyl-3′-N-propargyl)desosaminyl-6-O-methyl erythronolide A (Compound No. 7),
3-O-(3-Fluorophenyl)acetyl-5-O-(3′-N-desmethyl-3′-N-propargyl)desosaminyl-6-O-methyl erythronolide A (Compound No. 8),
3-O-(2-Fluorophenyl)acetyl-5-O-(3′-N-desmethyl-3′-N-allyl)desosaminyl-6-O-methyl erythronolide A (Compound No. 9),
3-O-(4-Fluorophenyl)acetyl-5-O-(3′-N-desmethyl-3′-N-(4-nitro) benzyl)desosaminyl-6-O-methyl erythronolide A (Compound No. 10),
3-O-(4-Nitrophenyl)acetyl-5-O-(3′-N-desmethyl-3′-N-ethyl)desoaminyl-6-O-methyl erythronolide A (Compound No. 11),
3-O-(2-Pyridyl)acetyl-5-O-(3′-N-desmethyl-3′-N-cyclopropylmethyl)desosaminyl-6-O-methyl erythronolide A (Compound No. 12),
3-O-(2-Naphthyl)acetyl-5-O-(3′-N-desmethyl-3′-N-benzyl)desosaminyl-6-O-methyl erythronolide A (Compound No. 13),
3-O-(2,4-Difluorophenyl)acetyl-5-O-(3′-N-desmethyl-3′-N-benzyl) desosaminyl-6-O-methyl erythronolide A (Compound No. 14),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyrrolo-(2,3-b)-pyridin-1-yl)-butyl)-imino)]erythromycin A (Compound No. 15),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(3H)-imidazol-[4,5-b]pyridin-3-yl)-butyl)-imino]erythromycin A (Compound No. 16),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(1H)-imidazol-[4,5-b]pyridin-1-yl)-butyl)-imino]-erythromycin A (Compound No. 17),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-pyrrolo-[2,3-b]pyridin-T-yl)-butyl)-imino]erythromycin A (Compound No. 18),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-indol-1-yl)-butyl)-imino]erythromycin A (Compound No. 19),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A (Compound No. 20),
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-quinolin-4-ylmethyl)-2-aminoethyl)-imino]erythromycin A (Compound No. 21),
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A (Compound No. 22),
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N-pyridine-4-ylmethyl)-2-aminoethyl)-imino]erythromycin A (Compound No. 23),
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A (Compound No. 24),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 25),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-nitro)-indol-1-yl)-butyl)-imino)]erythromycin A (Compound No. 26),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((1H)-imidazo[4,5-c]pyridin-1-yl)-butyl)-imino)]erythromycin A (Compound No. 27),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-2-yl)-butyl)-imino)]erythromycin A (Compound No. 28),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((3H)-imidazo-[4,5-c]pyridin-3-yl)-butyl)-imino)]erythromycin A (Compound No. 29),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-4-yl)-butyl)-imino)]erythromycin A (Compound No. 30),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-3-yl)-butyl)-imino)]erythromycin A (Compound No. 31),
2α-Fluoro-5-O-(3′-N-desmethyl-3′-N-allyl)-11,12-dideoxy-3-O-decladinosyl-6-O-methyl-3-oxo-12,11-[oxycarbonyl-((4-(benzotriazol-1-yl)-butyl)-imino)]erythromycin A (Compound No. 32),
2α-Fluoro-5-O-(3′-N-desmethyl-3′-N-allyl)-11,12-dideoxy-3-O-decladinosyl-6-O-methyl-3-oxo-12,11-[oxycarbonyl-((4-(benzoimidazol-1-yl)-butyl)-imino)]erythromycin A (Compound No. 33),
2α-Fluoro-5-O-(3′-N-desmethyl-3′-N-allyl)-11,12-dideoxy-3-O-decladinosyl-6-O-methyl-3-oxo-12,11-[oxycarbonyl-((4-((3H)-imidazo-[4,5-b]pyridin-3-yl)-butyl)-imino)]erythromycin A (Compound No. 34),
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 35),
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-(benzotriazol-2-yl)-butyl)-imino)]erythromycin A (Compound No. 36),
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-(indol-1-yl)-butyl)-imino)]erythromycin A (Compound No. 37),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-2-ylmethyl)-2-aminoethyl)-imino]erythromycin A (Compound No. 38),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-quinolin-4-ylmethyl)-2-aminoethyl)-imino]erythromycin A (Compound No. 39),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-4-ylmethyl)-2-aminoethyl)-imino]erythromycin A (Compound No. 40),
2α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A (Compound No. 41),
2α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A (Compound No. 42),
2α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-quinoline-4-ylmethyl)-2-aminoethyl)-imino]erythromycin A (Compound No. 43),
5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyridin-3-yl-methyl-benzimidazol-1-yl)-butyl)-imino)]erythromycin A (Compound No. 44),
5-O-(3′-N-desmethyl-3″-N-ethyl)desosaminyl-11,12-dideoxy-3-O-decladinosyl-6-O-methyl-3-oxo-12,11-[oxycarbonyl-((4-(4-pyridin-3-yl-imidazol-1-yl)-butyl)-imino)]erythromycin A (Compound No. 45),
5-O-(3′-N-desmethyl-3′-N-ethyl)desosaminyl-11,12-dideoxy-3-O-decladinosyl-6-O-methyl-3-oxo-12,11-[oxycarbonyl-((4-(imidazo[4,5-b]pyridin-1-yl)-butyl)-imino)]erythromycin A (Compound No. 46),
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-((5-nitro)-indol-1-yl)-butyl)-imino)]erythromycin A (Compound No. 47),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-fluoro)-indol-1-yl)-butyl)-imino)]erythromycin A (Compound No. 48),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-1-yl)-butyl)-imino)]erythromycin A (Compound No. 49),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A (Compound No. 50),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-2-yl)-butyl)-imino)]erythromycin A (Compound No. 51),
2α-Fluoro-5-O-(3′-N-desmethyl-3′-N-allyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A (Compound No. 52),
2α-Fluoro-5-O-(3′-N-desmethyl-3′-N²-fluoroethyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A (Compound No. 53),
2α-Fluoro-5-O-(3′-N-desmethyl-3′-N-allyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 54),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-pyridin-3-yl)-imidazol-1-yl)-butyl)-imino]erythromycin A (Compound No. 55),
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 56),
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-(benzotriazol-1-yl)-butyl)-imino)]erythromycin A (Compound No. 57),
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-pyridin-3-yl)-imidazol-1-yl)-butyl)-imino)]erythromycin A (Compound No. 58),
2α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 59),
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A (Compound No. 60),
2α-Fluoro-5-O-(3′-N-desmethyl-3′-N-allyl)-11,12-dideoxy-3-O-decladinosyl-6-O-methyl-3-oxo-12,11-[oxycarbonyl-((N′-acetyl-N′-pyridine-3-ylmethyl)-2-aminoethyl)-imino]erythromycin A (Compound No. 61),
2α-Fluoro-5-O-(3′-N-desmethyl-3′-N-allyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A (Compound No. 62),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(3H)imidazo[4,5-b]pyridin-3-yl)-butyl)-imino]erythromycin A (Compound No. 63),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-phenyl)-imidazol-1-yl)-butyl)-imino]erythromycin A (Compound No. 64),
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-(3H)imidazo[4,5-b]pyridin-3-yl)-butyl)-imino]erythromycin A (Compound No. 65),
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A (Compound No. 66),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(quinolin-8-yl)-butyl)-imino)]erythromycin A (Compound No. 67),
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A (Compound No. 68),
2α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-pyridin-3-yl)-imidazo)-1-yl)-butyl)-imino)]erythromycin A (Compound No. 69),
11,12-dideoxy-3-O-decladinosyl-3-O-(2-pyridylacetyl)-5-O-(3′-N-desmethyl-3′-N-propargyl)-6-O-methyl-12,11-[oxycarbonyl-((4-benzoimidazo-1-yl)-butyl)-imino]erythromycin A (Compound No. 70),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A (Compound No. 71),
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A (Compound No. 72),
2α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyridin-2-yl-1H-imidazol-1-yl)-butyl)-imino))]erythromycin A (Compound No. 73),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl-pyrazol-1-yl)-butyl)-imino))erythromycin A (Compound No. 74),
2α-Fluoro-5-O-(3′-N-desmethyl-3′-N-allyl)-11,12-dideoxy-3-O-decladinosyl-6-O-methyl-3-oxo-12,11-[oxycarbonyl-((3-pyrrolo[2,3-b]pyridin-1-yl)-propyl)-hydrazo)]erythromycin A (Compound No. 75),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((indazol-2-yl)-butyl)-imino))]erythromycin A (Compound No. 76),
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-amino-9H-purin-9-yl butylimino)]erythromycin A (Compound No. 77),
11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(isoquinolin-5-yl)-butyl)-imino)]erythromycin A (Compound No. 78),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A (Compound No. 79),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A (Compound No. 80),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-allyl-3-(9-(4-amino-butyl)-9H-purin-6-yl)-urea)-imino)]erythromycin A (Compound No. 81),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-2-yl-1H-imidazol-lyl)-butyl)-imino)]erythromycin A (Compound No. 82),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(1H-imidazol[4,5-c]pyridin-1-yl)-butyl)-imino)]erythromycin A (Compound No. 83),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-3-yl)-1H-imidazol-1-yl)-propyl)-hydrazo)]erythromycin A (Compound No. 84),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-purin-9-yl)-propyl)-hydrazo)]erythromycin A (Compound No. 85),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-purin-9-yl)-propyl)-hydrazo)]erythromycin A (Compound No. 86),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-isoquinolin-5-yl)-propyl)-hydrazo)]erythromycin A (Compound No. 87),
2α-Fluoro-5-O-(3′-N-desmethyl-3′-N-ethyl)-11,12-dideoxy-3-O-decladinosyl-6-O-methyl-3-oxo-12,11-[oxycarbonyl-hydrazo]erythromycin A (Compound No. 88),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(isoquinolin-5-yl)-butyl)-imino)]erythromycin A (Compound No. 89),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-imidazol-lyl)-butyl)-imino)]erythromycin A (Compound No. 90),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-3-yl-1H-imidazol-lyl)-butyl)-imino)]erythromycin A (Compound No. 91),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-furan-3-yl-1H-imidazol-lyl)-butyl)-imino)]erythromycin A (Compound No. 92),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A (Compound No. 93),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A (Compound No. 94),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-2-yl-1H-imidazol-1-yl)-propyl)-hydrazo)]erythromycin A (Compound No. 95),
2α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol-1-yl)propyl)-hydrazo)]erythromycin A (Compound No. 96),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-furan-2-yl-1H-imidazol-1-yl)-butyl)-imino))]erythromycin A (Compound No. 97),
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-thiophen-2-yl-1H-imidazol-1-yl)-butyl)-imino))]erythromycin A (Compound No. 98),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-2-yl)-pyrazol-1-yl)-butylimino)]erythromycin A (Compound No. 99),
11,12-dideoxy-3-O-decladinosyl-3-O-(2-pyridyl acetyl)-5-O-(3′-N-desmethyl-3′-N-allyl)-6-O-methyl-12,11-[oxycarbonyl-4-([1,4′]-bipyrazol-1′-yl)-butylimino)]erythromycin A (Compound No. 100),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-1-yl)-propyl)-hydrazo)]erythromycin A (Compound No. 101),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-3-yl)-propyl)-hydrazo)]erythromycin A (Compound No. 102),
2α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-thiophen-3-yl-phenoxy)-propyl)-imino)]erythromycin A (Compound No. 103),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(3-thiophen-3-yl-phenoxy)-propyl)-imino)]erythromycin A (Compound No. 104),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-thiazolyl)-imidazol-1-yl)-butylimino)]erythromycin A (Compound No. 105),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiazol-2-yl)-imidazol-1-yl)-butylimino)]erythromycin A (Compound No. 106),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A (Compound No. 107),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A (Compound No. 108),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A (Compound No. 109),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A (Compound No. 110),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(1H-imidazol[4,5-b]pyridin-2-yl)-butylimino)]erythromycin A (Compound No. 111),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-tetrazol-1-yl)-imidazol-1-yl)-butylimino)]erythromycin A (Compound No. 112),
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-Imidazo[4,5-b]pyridine-2-yl)-butyl)-imino)erythromycin A (Compound No. 113),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(1H-benzimidazol-2-yl)-butylimino)]erythromycin A (Compound No. 114),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A (Compound No. 115),
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Tetrazol-1-yl-imidazol-1-yl)-butyl)-imino)erythromycin A (Compound No. 116),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A (Compound No. 117),
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-Benzoimidazol-2-yl)-butyl)-imino)erythromycin A (Compound No. 118),
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(4-imidazol[4,5-b]pyridin-1-yl)-butylimino)]erythromycin A (Compound No. 119),
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(6-amino-9H-purin-9-yl)butylimino)]erythromycin A (Compound No. 120),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[4-(pyrazol-1-yl)-imidazol-1-yl)butylimino]erythromycin A (Compound No. 121),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-([1,4′]-biimidazol-1′-yl)butylimino)]erythromycin A (Compound No. 122),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([1,4′]-biimidazol-1′-yl)butylimino)]erythromycin A (Compound No. 123),
11,12-dideoxy-3-O-decladinosyl-3-O-(2-methylpropanoyl)-5-O-(3′-N-desmethyl-3′-N-allyl)-6-O-methyl-12,11-[oxycarbonyl-(4-(imidazo[4,5-b]pyridin-3-yl)butylimino)]erythromycin A (Compound No. 124),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyrazol-1-yl)-imidazol-1-yl)butylimino)]erythromycin A (Compound No. 125),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A (Compound No. 126),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A (Compound No. 127),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiazol-2-yl)butylimino)]erythromycin A (Compound No. 128),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(4-pyrazol-1-yl)-imidazol-1-yl)-propyl)-hydrazo]erythromycin A (Compound No. 129),
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-(1H-imidazo[4,5-b]pyridine-1-yl)-propyl)-hydrazo)]erythromycin A (Compound No. 130),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(oxazol-5-yl)-imidazol-1-yl)butylimino)]erythromycin A (Compound No. 131),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A (Compound No. 132),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A (Compound No. 133),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[3,3′]-bithiophenyl-5-yl)butylimino)]erythromycin A (Compound No. 134),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-3-yl)-tetrazol-2-yl)butylimino)]erythromycin A (Compound No. 135),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(6-chloropyridin-3-yl)-imidazol-1-yl]butylimino)]erythromycin A (Compound No. 136),
11,12-dideoxy-3-O-decladinosyl-3-O-(2-methylpropanoyl)-5-O-(3′-N-desmethyl-3′-N-allyl)-6-O-methyl-12,11-[oxycarbonyl-4-(4-(4-(furan-2-yl)-imidazol-1-yl)-butylimino)]erythromycin A (Compound No. 137),
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-([1,4′]-bipyrazol-1-yl)-)-propyl)-hydrazo)]erythromycin A (Compound No. 138),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(2-methoxy-phenyl)-imidazol-1-yl)-butylimino)]erythromycin A (Compound No. 139),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-methoxy-phenyl)-1H-imidazol-1-yl)-butylimino)]erythromycin A (Compound No. 140),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A (Compound No. 141),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A (Compound No. 142),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-furan-2-yl-thiophen-2-yl)butylimino)]erythromycin A (Compound No. 143),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-furan-2-yl-thiophen-2-yl)-butylimino)]erythromycin A (Compound No. 144),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A (Compound No. 145),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A (Compound No. 146),
11,12-dideoxy-3-O-decladinosyl-3-O-(2-nitrophenyl acetyl)-5-O-(3′-N-desmethyl-3′-N-allyl)-6-O-methyl-12,11-[oxycarbonyl-(4-benzimidazol-1-yl)butylimino)]erythromycin A (Compound No. 147),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(4-pyrazol-1-yl]-imidazol-1-yl)-propyl)-hydrazo)]erythromycin A (Compound No. 148),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(3-aminophenyl)-imidazol-1-yl)-butylimino)]erythromycin A (Compound No. 149),
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Thiophen-3-yl-pyrazol-1-yl)-butyl)-imino)erythromycin A (Compound No. 150), or
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Thiophen-2-yl-pyridin-3-yl)-butyl)-imino)-erythromycin A (Compound No. 151),
   as well as pharmaceutically acceptable salts, pharmaceutically acceptable solvates, polymorphs, prodrugs, stereoisomers, tautomeric forms, N-oxides and metabolites thereof.

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)R_{f}, —NR_{f}R_{q}, —C(═O)NR_{f}R_{q}, —NHC(═O)NR_{f}R_{q}, —C(═O)heteroaryl, C(═O)heterocyclyl, 0-CC(═O)NR_{f}R_{q} {wherein R_{f} and R_{q} are independently selected from hydrogen, alkyl, alkenyl, cycloalkyl, cycloalkenyl, aryl, aralkyl, heterocyclyl, heteroaryl, heterocyclylalkyl, heteroarylalkyl}, nitro, or —SO₂Rⱼ (wherein Rⱼ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, carboxy, —NR_{f}R_{q}, —C(═O)NR_{f}R_{q}, —OC(═O)NR_{f}R_{q}, —NHC(═O)NR_{f}R_{q} (wherein R_{f} and R_{q} are the same as defined earlier), hydroxy, alkoxy, halogen, CF₃, cyano, and —SO₂R_{b}, (wherein R_{b} is alkyl, alkenyl, alkynyl, cycloalkyl, aralkyl, aryl, heterocyclyl, heteroaryl, heteroarylalkyl or heterocyclylalkyl); or an alkyl group also may be interrupted by 1-5 atoms of groups independently selected from oxygen, sulfur or —NRₐ— {wherein R₁ is selected from hydrogen, alkyl, cycloalkyl, alkenyl, cycloalkenyl, alkynyl, aryl, acyl, aralkyl, —C(═O)OR_{f} (wherein R_{f} is the same as defined earlier), SO₂R₄ (where R₄ is as defined earlier), or —C(═O)NR_{f}R_{q} (wherein R_{f} and R_{q} are as defined earlier)}. Unless otherwise constrained by the definition, all substituents may be substituted further by 1-3 substituents selected from alkyl, carboxy, —NR_{f}R_{q}, —C(═O)NR_{f}R_{q}, —O—C(═O)NR_{f}R_{q}, NHCOOR_{b} (wherein R_{f}, R_{q} and R_{b} are the same as defined earlier), hydroxy, alkoxy, halogen, CF₃, cyano, and —SO₂Rⱼ (where Rⱼ is same as defined earlier); or an alkyl group as defined above that has both substituents as defined above and is also interrupted by 1-5 atoms or groups as defined above.

The term “alkenyl or alkynyl” stands for unsaturated hydrocarbon having two to six carbon atoms. One or more hydrogen of said alkenyl or alkynyl can be replaced by halogen, NHCOR_{b}, NHCOOR_{b}, COR_{b}, OCOR_{b} (wherein R_{b} is the same as defined earlier). Examples of alkenyl and alkynyl include, but are not limited to, ethylene, propylene, ethynyl, propynyl, and the like.

The term “alkoxy” stands for a radical represented by Formula O-alkyl and wherein alkyl is the same as defined above. Examples of alkoxy include, but are not limited to, methoxy, ethoxy, propoxy, isopropoxy, and the like.

The term “cycloalkyl” refers to saturated carbocyclic ring having three to seven carbon atoms. Examples of cycloalkyl include, but are not limited to, cyclopropyl, cyclobutyl and cyclopentyl, and the like.

The term “halogen” refers to fluorine, chlorine, bromine or iodine.

The term “aryl,” unless otherwise specified, refers to carbocyclic aromatic groups, for example, phenyl, biphenyl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₑ (wherein Rₑ is hydrogen, alkyl, alkenyl, cycloalkyl, aralkyl, heterocyclylalkyl, heteroarylalkyl), NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), —SO₂R_{b} (wherein R_{b} is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S.

The term “heteroaryl,” unless otherwise specified, refers to an aromatic ring structure containing 5 or 6 ring atoms, or a bicyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{f}R_{q}, CH═NOH, —(CH₂)_{w}C(═O)R_{g} {wherein w is an integer from 0-4 and R_{g} is hydrogen, hydroxy, OR_{f}, NR_{f}R_{q}, —NHOR_{z} or —NHOH}, —C(═O)NR_{f}R_{q} and —NHC(═O)NR_{f}R_{q}, —SO₂R_{b}, —O—C(═O)NR_{f}R_{q}, —O—C(═O)R_{f}, —O—C(═O)OR_{f} (wherein R_{b}, R_{f} and R_{q} are as defined earlier, and R_{z} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzothiazolyl, or benzoxazolyl, and the like.

The term ‘heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, aryl, alkoxy, alkaryl, cyano, nitro, oxo, carboxy, heterocyclyl, heteroaryl, —O—C(═O)R_{f}, —O—C(═O)OR_{f}, —C(═O)NR_{f}R_{q}, SO₂R_{b}, —O—C(═O)NR_{f}R_{q}, —NHC(═O)NR_{f}R_{q}, —NR_{f}R_{q} (wherein R_{b}, R_{f} and R_{q} are as defined earlier) or guanidine. Heterocyclyl can optionally include rings having one or more double bonds. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, dihydropyridinyl, dihydroisoxazolyl, dihydrobenzofuryl, azabicyclohexyl, dihydroindolyl, pyridinyl, isoindole 1,3-dione, piperidinyl or piperazinyl.

The groups “alkyl, aryl, heteroaryl and heterocyclyl” can optionally be substituted with substituent(s) selected from alkyl, haloalkyl, cycloalkyl, cycloalkenyl, cycloalkoxy, aryl, heterocyclyl, heteroaryl, aralkyl, heteroarylalkyl, heterocycloalkyl, halogen, hydroxy, alkoxy, cyano, nitro, aryloxy, haloalkoxy, COR_{b}, CSR_{b}, COOR_{b}, S(O)ₐR_{b}, OCOOR_{b}, OCOR_{b}, SO₂NHR_{b}, NHSO₂R_{b}, NHCOR_{b}, NHCSR_{b}, (CH)ₐC(═O)NR_{c}R_{d} or NR_{c}R_{d} (wherein R_{c}, and R_{d} are independently selected from hydrogen, alkyl, aryl, heteroaryl, heterocyclyl, R_{b} is the same as defined earlier and a is an integer of from 0-2). Unless otherwise constrained, all substituents may optionally be further substituted by substituent(s) defined earlier.

The term “polymorphs” includes all crystalline form as well as amorphous form for compounds described herein and as such are encompassed herein.

The term “pharmaceutically acceptable carriers” is intended to include non-toxic, inert solid, semi-solid or liquid filler, diluent, encapsulating material or formulation auxiliary of any type.

The term “pharmaceutically acceptable salts” refer to a salt prepared from pharmaceutically acceptable organic or inorganic acids; such salts include hydrochlorides, sulfates, phosphates, tartarates, fumarates, citrates and the like. The free base forms of compounds described herein may be prepared from the salt forms, if desired, by contacting the salt with dilute aqueous solution of a base. The acid addition salts may differ from the free base forms of the compounds described herein in such physical characteristics as solubility and melting point.

The salt forms differ from the compound described herein in certain physical properties such as solubility, but the salts are otherwise equivalent for purposes herein.

The term “pharmaceutically acceptable” means approved by regulatory agency of the federal or a state government or listed in the U.S. Pharmacopoeia or other generally recognized pharmacopoeia for use in animals, and more particularly in humans.

The term “pharmaceutically acceptable solvates” refers to solvates with water (i.e., hydrates, hemihydrate or sesquihydrate) or pharmaceutically acceptable solvents, for example solvates with common organic solvents as ethanol and the like. Such solvates are also encompassed within the scope of the disclosure.

Also encompassed herein are “prodrugs” of compounds described herein. In general, such prodrugs will be functional derivatives of these compounds, which are readily convertible in vivo into the required compound. They may be carrier-linked or bioprecursors. The carrier-linked prodrugs may be bipartite, tripartite or mutual prodrugs. Prodrugs are intended to improve drug efficacy by improving solubility and consequently absorption and distribution as desired. Conventional procedure for the selection and preparation of suitable prodrug derivatives are described, for example, in “Design of Prodrugs”, ed. H Bundgaard and, Elsevier, 1985. Enantiomers and Diastereomers, are as defined by the IUPAC 1974 Recommendations for Section E.

In another aspect, provided herein are pharmaceutical compositions comprising therapeutically effective amounts of one or more compounds of Formula I, or pharmaceutically acceptable salts, pharmaceutically acceptable solvates, stereoisomers, polymorphs, N-oxide, prodrugs or metabolites thereof, together with one or more pharmaceutically acceptable carriers, excipients, diluents or mixtures thereof. These compositions can be administered by any route of administration, for example, orally, sublingually, parenterally, topically, nasally, rectally or transdermally for treating or preventing inflammatory diseases or disorders.

In one embodiment, provided herein are pharmaceutical compositions comprising therapeutically effective amounts of one or more compounds of Formula Ia, or pharmaceutically acceptable salts, pharmaceutically acceptable solvates, stereoisomers, polymorphs, N-oxide, prodrugs or metabolites thereof, together with one or more pharmaceutically acceptable carriers, excipients, diluents or mixtures thereof.

In another embodiment, provided herein are pharmaceutical compositions comprising therapeutically effective amounts of one or more compounds of Formula Ib, or pharmaceutically acceptable salts, pharmaceutically acceptable solvates, stereoisomers, polymorphs, N-oxide, prodrugs or metabolites thereof, together with one or more pharmaceutically acceptable carriers, excipients, diluents or mixtures thereof.

In another embodiment, provided herein are pharmaceutical compositions comprising therapeutically effective amounts of one or more compounds of Formula 2, pharmaceutically acceptable salts, pharmaceutically acceptable solvates, stereoisomers, polymorphs, N-oxide, prodrugs or metabolites thereof, together with one or more pharmaceutically acceptable carriers, excipients, diluents or mixtures thereof.

Compounds described herein can be administered to a patient (e.g., human or animal) by any route of administration, for example, orally, parenterally, topically, rectally, intranasally, subcutaneously or transdermally.

Suitable solid dosage forms include, for example, tablets, capsules, microcapsules, powders, granules, pills, wafers, dragees, catchets, caplets, troches, suppositories and pastilles.

Pharmaceutical compositions for solid dosage forms may contain one or more agents selected from a) fillers, such as dibasic calcium phosphate, calcium carbonate, dextrose, sucrose, glucose, mannitol, silicic acid, starch, lactose or mixtures thereof, b) lubricants, such as talc, sodium lauryl sulfate, calcium stearate, simethicone, stearic acid, zinc stearate, magnesium stearate or mixtures thereof, c) binders, such as carboxymethyl cellulose, alginate, gelatin, polyvinylpyrolidone, gum acacia, agar, guar gum, microcrystalline cellulose, sorbitol, tragacanth or mixtures thereof, d) glidants, such as colloidal silicon dioxide; e) disintegrating agents, such as agar-agar, calcium carbonate, sodium carbonate, alginic acid, tapioca, potato starch or mixtures thereof, f) wetting agents, such as acetyl alcohol, glycerol monostearate or mixtures thereof, g) sweetener, such as fructose, mannitol, sorbitol, xylitol or mixtures thereof, h) humectants, such as glycerol; or i) mixtures thereof.

Tablets, capsules, pills are generally administered as a unit dose and may contain one or more suitable excipients, such as dispersing agents, binding agents, fillers, diluents, lubricants, disintegrants, colorants, flavoring agents, sweeteners, preservatives or mixtures thereof.

Tablets, pills, dragees, capsules and granules may be sugar coated, enteric-coated film coated and other coatings well known in the pharmaceutical formulating art. Immediate release tablets and tablets having sustained action may also be prepared by any method known in the art. Capsules may be hard capsules or soft capsules of suitable size.

Dispersible powders and granules suitable for reconstitution to form a stable suspension by addition of water are provided with the active ingredient, one or more dispersing agents and one or more suspending agents. Additional excipients, for example, coloring agents, flavoring agents or sweetening agents may also be added.

Suppositories for rectal administration may include carbon dioxide-releasing laxative suppositories, which dosage form for topical or transdermal administration includes ointments, pastes, creams, lotions, gels, powders, solutions, sprays, inhalants, spot-on or patches. Active compounds can be admixed under sterile condition with one or more pharmaceutically acceptable carriers and any optional preservatives or buffers as may be desired.

Liquid form preparations for oral administration includes pharmaceutically acceptable emulsions, microemulsions, solutions, aqueous or oily suspensions, syrups, sprays or elixirs.

Pharmaceutical compositions as emulsions may be in the form of oil in water, water in oil or multiple emulsions, e.g., oil/water/oil, water/oil/water for oral or topical administration. The oily phase may comprise arachis, olive, groundnut, corn, germ, castor, sesame, mineral oil e.g., liquid paraffin or mixtures thereof. Liquid dosage forms may contain one or more agents selected from emulsifying agents, solubilizers, such as gum acacia, egg lecithin, gum tragacanth, sorbitan esters, propylene glycol, ethyl acetate, oil and mixtures thereof; perfuming agents, flavoring agents, preservatives, such as methyl paraben, ethyl paraben or mixture thereof; sweetening agents, wetting agents with a suitable HLB value e.g., as given above.

Suspensions may be oily or aqueous. Suspensions may contain one or more agents selected from a) suspending agents, such as tragacanth, acacia, colloidal silicon dioxide, sodium carboxymethylcellulose, methylcellulose, carbomer resins, hydroxymethylcellulose, polyvinylpyrrolidone, sodium alginate or mixtures thereof, b) dispersing or wetting agents, such as lecithin; c) suspending agents or thickening agents, such as beeswax, hard paraffin, cetyl alcohol, sorbitol, syrup, methyl cellulose, gelatin, hydroxyethylcellulose, carboxymethylcellolose or mixtures thereof; d) preservatives, such as methyl paraben, ethyl paraben, sorbic acid and the like or mixtures thereof. Other suitable agents, such as perfuming agents, flavoring agents or sweetening agents, may be included.

For liquid form preparations, active compounds can be mixed with water and/or one or more other solvents, solubilizing agents, emulsifiers, for example, ethyl alcohol, isopropanol, ethyl carbonate, ethyl acetate, benzyl alcohol, benzyl benzoate, propylene glycol, 1,3-butylene glycol, dimethylformamide, oils, for example, cottonseed, groundnut, corn, germ, olive, castor and sesame oil, glycerol, and fatty acid esters of sorbitan and mixtures thereof. Spray compositions can contain one or more suitable propellants.

Injectable preparations, for example, sterile aqueous or non-aqueous injections, injectable depot forms, aqueous suspensions or emulsions may be formulated according to the art using parenterally dispersing or wetting agents and/or suspending agents. Among the acceptable vehicles and solvents that may be employed are water for injection, Ringer's solution and isotonic sodium chloride. Suitable route of administration may include intravenous, intramuscular, subcutaneous, intrasternal or infusion.

Ophthalmic formulations, eardrops, eye ointments, powders and solutions are also encompassed.

Dosage forms for topical or transdermal administration of compound s described herein include ointments, pastes, creams, lotions, gels, powders, solutions, sprays, inhalants or patches. Active compounds can be admixed under sterile conditions with one or more pharmaceutically acceptable carriers, as well as preservatives or buffers as may be desired. Pharmaceutically acceptable carriers can be selected from lactose, talc, silicates, aluminium hydroxide and mixtures thereof.

Aerosols for nasal administration can be prepared according to the techniques known in the art. Aerosols may contain one or more suitable preservatives, anti-oxidants, dispersing agents and the like or mixtures thereof. Aerosol devices used can include, for example, jet, vibrating porous plate, ultrasonic nebulizers or metered dose inhalers.

Pharmaceutical preparations can be in unit dosage form. Such preparations can be subdivided into unit doses containing appropriate quantities of the active component. The unit dosage form can be packaged preparation, the package containing discrete capsules, powders, in vials or ampoules, and ointments capsule, sachet, tablet, gel, cream itself or it can be the appropriate number of any of these packaged forms.

Therapeutically effective doses for any compound or pharmaceutical composition disclosed herein varies with the gravity of the symptoms and can be determined initially from the animal models. Taking the animal dosing into consideration, one of ordinary skill in the art can readily determine the optimum doses in mammals without undue experimentation.

In another aspect, provided herein are combinations of one or more compounds described herein with one or more other anti-inflammatory agents. Such combinations can be used to treat or prevent inflammatory diseases or disorders and can achieve additive or synergistic effects. These other anti-inflammatory agents may include steroidal anti-inflammatory agents; non-steroidal anti-inflammatory agents; COX-2 inhibitors; macrolide immunosuppresives, p-38 MAP Kinase inhibitors; local anesthetics; beta-2 agonists; 5-lipooxygenase inhibitors; phosphodiesterase inhibitors; MMP inhibitors; TNF-alpha inhibitors; caspase inhibitors; VLA4 antagonists; PAF antagonists; or mixtures thereof.

In another aspect, provided herein are combination pharmaceutical compositions for treating or preventing inflammatory diseases or disorders. The term “combination pharmaceutical compositions,” as used herein, can refer to the following:
a) a single pharmaceutical composition comprising one or more compounds of Formula I and one or more of steroidal anti-inflammatory agents; non-steroidal anti-inflammatory agents; COX-2 inhibitors; macrolide immunosuppresives, p-38 MAP Kinase inhibitors; local anesthetics; beta-2 agonists; 5-lipooxygenase inhibitors; phosphodiesterase inhibitors; MMP inhibitors; TNF-alpha inhibitors; caspase inhibitors; VLA4 antagonists; PAF antagonists or mixture thereof, optionally together with one or more pharmaceutically acceptable carriers, excipients, diluents or mixture thereof.
b) a first pharmaceutical composition comprising therapeutically effective amount of at least one macrolide derivatives of Formula I, together with one or more pharmaceutically acceptable carriers, excipients, diluents or mixture thereof; a second pharmaceutical composition comprising one or more of steroidal anti-inflammatory agents; non-steroidal anti-inflammatory agents; COX-2 inhibitors; macrolide immunosuppresives, p-38 MAP Kinase inhibitors; local anesthetics; beta-2 agonists; 5-lipooxygenase inhibitors; phosphodiesterase inhibitors; MMP inhibitors; TNF-alpha inhibitors; caspase inhibitors; VLA4 antagonists; PAF antagonists or mixture thereof, together with pharmaceutically acceptable carriers, excipients, diluents or mixture thereof. The separate pharmaceutical composition can be administered simultaneously, separately or sequentially in any order.

Suitable anti-inflammatory agents include steroidal anti-inflammatory agents; non-steroidal anti-inflammatory agents; COX-2 inhibitors; macrolide immunosuppresives, p-38 MAP Kinase inhibitors; local anesthetics; beta-2 agonists; 5-lipooxygenase inhibitors; phosphodiesterase inhibitors; MMP inhibitors; TNF-alpha inhibitors; caspase inhibitors; VLA4 antagonists; PAF antagonists or mixtures thereof. Such anti-inflammatory agents may be widely chosen from among those already known in the prior art or subsequently discovered and/or hereafter discovered and/or hereafter developed.

Suitable steroidal anti-inflammatory agents can be selected from, but are not limited to, alclometasone, amcinonide, amelometasone, beclometasone, betamethasone, budesonide, ciclesonide, clobetasol, cloticasone, cyclomethasone, deflazacort, deprodone, dexbudesonide, diflorasone, difluprednate, fluticasone, flunisolide, halometasone, halopredone, hydrocortisone, hydrocortisone, methylprednisolone, mometasone, prednicarbate, prednisolone, rimexolone, tixocortol, triamcinolone, ulobetasol, their pharmaceutically acceptable salts, solvates thereof, or mixtures thereof.

Suitable non-steroidal anti-inflammatory agents can be selected from, but are not limited to, salicylic acids (e.g., aspirin or diflunisal), propionic acids (e.g., ketoprofen or naproxen), acetic acids (e.g., diclofenac or indomethacin), enolic acids (e.g., piroxicam or meloxicam), fenamic acids (e.g., mefenamic acid or meclofenamate), napthylalkanones (e.g., nabumetone), pyranocarboxylic acids (e.g., etodalac), pyrroles (e.g., ketorolac), COX-2 inhibitors (e.g., valdecoxib, celecoxib, parecoxib or rofecoxib) or mixtures thereof.

β2-agonists can be selected from, but are not limited to, albuterol, formoterol, terbutaline, metaproterenol or mixtures thereof. 5-lipooxygenase inhibitors can be selected from, but are not limited to, zileuton, atreluton or a mixture thereof. Phosphodiesterase IV inhibitors can be selected from, but are not limited to, RBx-11082, cilomilast, roflumilast or mixtures thereof. MMP inhibitors can be selected from, but are not limited to, batimastat (BB-94), marimastat (BB-2516), prinomastat (AG3340), BAY 12-9566, CGS27023A or mixtures thereof. TNF-α inhibitors can be selected from, but are not limited to, infliximab, etanercept, D2E7, CDP 571 or mixtures thereof. Caspase inhibitors can be selected from, but is not limited to, pralnacasan (Vx-740). p38 map kinase inhibitors can be selected from, but are not limited to, Vx-745, BIRB-796, RWJ-67657, SB-239063 or mixtures thereof. VLA-4 antagonists can be selected from, but are not limited to, clafrinast, RBx-7796 or mixture thereof. Platelet-activating factor (PAF) antagonists can be selected from, but are not limited to, apafant, ibudilast, lexipafant, rupatadine, ginkgolides (e.g., ginkgolide A, B or C), derivatives thereof or mixtures thereof.

The term “inflammatory disease” as used herein refers to any disease, disorder or condition associated with inflammation. The disease, disorder or condition may arise due to any exogenous or endogenous factors, except infection caused by organisms, such as bacteria, viruses, fungi, protozoa and the like. Examples of inflammatory disease include, for example, respiratory diseases viz. bronchitis, bronchiectasis, rhinitis, cystic fibrosis, emphysema, asthma, sinusitis, tonsillitis, chronic obstructive pulmonary disease, interstitial lung fibrosis or adult respiratory distress syndrome; renal diseases viz. glomerulonephritis (with and without nephritic syndrome); autoimmune diseases viz., rheumatoid arthritis, arthritis chronica progrediente, arthritis deformans, gout, autoimmune thyroiditis, autoimmune bullous dermatoses, multiple sclerosis, systemic lupus erythematosus, myasthenia gravis, haemolytic anaemia, aplastic anaemia, pure red cell anaemia, idiopathic thrombocytopenia; skin diseases viz., psoriasis, eczema, allergic dermatitis, neurodermatitis, scleroderma, dermatomyositis or pruritis; cardiovascular diseases viz., myocardial ischemia, atherosclerosis, or congestive heart failure; inflammatory bowel disease viz., Crohn's disease, ulcerative colitis, colitis, others which include, Reiter's syndrome, leukocytoclastic vasculitis, conjunctivitis, uveoretinitis, septic shock, rheumatoid spondylitis, osteoarthritis or septic arthritis. Inflammation associated with smoking, air pollution, other environmental factors and inflammation associated with organ transplant rejection is also encompassed herein.

### EXAMPLES

The following assays can be performed to show anti-inflammatory activity of compounds, compositions and combinations described herein. The following assays are illustrative and are not intended to limit the scope herein. Other assay methods known in the art can also be used.

### A. Cell Survival NFkB Assay:

J774A.1 were cultured in RPMI-1640 medium supplemented with 10% FBS, 2 mM L-glutamine, 100-units/mL penicillin and 100 g/mL streptomycin and seeded in a 96 well plate at a density of 0.2 million cells/well (in 180 μl of medium). The dilution of standard compound (BAY-11-7082) and compounds to be tested were made in DMSO and RPMI-1640 medium and 20 μl of each dilution is added to the cells. Plates were kept at 37° C. in 5% CO₂ incubator for 18 hours after shaking for 10 minutes. The effect of compounds on the death of cells was measured 18 hours post treatment of compounds. The cell viability was measured by MTT assay, which relies on the fact that viable cells converts the water soluble MTT (3-(4,5-dimethylthiazol-2-yl)-2,5-diphenyltetrazolium bromide) to an insoluble formazan salt. The formazan was then solubilized, and the concentration was determined by optical density at 540 nM. MTT solution 10 μL (5 mg/mL in RPMI medium) was added to each well and the cells were kept for 4 hours at 37° C. in 5% CO₂ incubator. Supernatant was discarded, pellet was dissolved in 100 μL of dimethyl sulphooxide (DMSO) and absorbance of the converted dye was measured at a wavelength of 540 nm. Cell survival was estimated as a percentage of the value of untreated controls.

Compounds herein that were tested exhibited NFkB-cell survival (as % inhibition at 10 μM) of about 75% to about 4%, from about 75% to about 30%, from about 75% to about 45% and even from about 75% to about 55%.

B. A23187 Induced LTB4 Release from Human Neutrophils

Neutrophils are isolated from freshly drawn human blood after dextran sedimentation and ficoll separation [Eur J. Biochem., 169, 175-184, (1987)]. 180 μL of the of neutrophil suspension (0.2×10⁶ cells/mL) is taken and added 19 μL of Hank's Buffer salt solution along with 1 μL of the test drug (200 times concentrated) in a 24 well plate and incubated at 37° C. for about 1 hour. 0.25 mM Ca⁺⁺/Mg⁺⁺ is added 3 minutes before the end of test compound incubation. Then, 0.3 μg/mL A23187 is added and incubated for further 10 minutes. The reaction is stopped by adding 80 μl of cold methanol and spun at 3500 rpm for 10 minutes to remove cell debris. The samples are analysed for LTB₄ assay [J. Pharmacol. Exp. Ther. 297, 1, 267-279, (2001)] using LTB₄ ELISA kits (Assay Design Inc., USA). The amount of LTB₄ is quantified and percent inhibition in LTB₄ release is calculated with respect to negative and positive control to compute IC₅₀ values.

### C. Assay for Matrix Metallo Proteinases

Test compounds/standards are prepared (stock 10 mM) in 100% DMSO and subsequent dilutions are made in 50% DMSO-50% TCNB (50 mM Tris, 10 mM CaCl₂, 150 mM NaCl, 0.05% Brij-35, pH 7.5). 1 μl of the compound and 88 μl of TCNB is added to wells of 96 well plate to achieve the desired final concentration of NCE (final DMSO concentration should not exceed 0.5%). 1 μL of activated, recombinant MMPs is added to each well (20-100 ng/100 μL reaction mixture) except the “negative well”. (MMP-1, 9 &14 enzymes require prior activation. For this, supplied enzyme was incubated with either APMA, final concentration 1 mM, for a time period of 1 hour at 37° C.). Incubation is done at room temperature for 4-5 min. Reaction is initiated with 10 μl of 100 μM substrate (ES001: Aliquots were freshly diluted in TCNB; stock: 2 mM) and increase in florescence is monitored at excitation wavelength 320 nm followed by emission at 405 nm for 25-30 cycles. Increase in florescence (RFU) is calculated for positive, negative and NCE/standard wells. The percent inhibition compared to controls is calculated and IC₅₀ values are determined by using Graph-prism software.

### D. Interleukin Converting Enzyme Inhibition Assays

The assay employs the calorimetric substrate Ac-YVAD-pNA that upon cleavage exhibits increased absorption at 405 nm, which is monitored in the presence and absence of inhibitor. Percent inhibition is calculated in a dose response curve and IC₅₀ values are calculated using PRISM-Graph Pad.

### E. Cell Based Assay for IL-1β Release

### (1) Method of Isolation of Human Peripheral Blood Mononuclear Cells:

Human whole blood is collected in vacutainer tubes containing EDTA as an anti coagulant. A blood sample (5 mL) is carefully extracted over 3 mL Ficoll Hypaque Cell Isolation Medium (Sigma) in a 15 mL round bottom centrifuge tubes. The sample is centrifuged at 450-500×g for 30-35 minutes in a swing out rotor at room temperature. After centrifugation the fluffy interface of cells is removed and is washed 3 times with phosphate buffered saline. The cells are centrifuged at 400×g for 10 minutes at room temperature. The cells are resuspended in RPMI media at concentration of 1 million cells/mL.

(2) LPS stimulation of Human Peripheral Blood Monocytes (PBM)

PBM cells (0.1 mL; 1 million/mL) are co-incubated with 0.1 mL of compound (10-0.41 μM, final concentration) for about 1 hour in flat bottom 96 well microtiter plate. Compounds are dissolved in DMSO initially and diluted in RPMI for a final concentration of 0.1% DMSO. Lipopolysaccharide (Cal biochem, 20 ng/mL, final concentration) is then added at volume of 0.010 mL. Cultures are incubated overnight at 37° C. Supernatant is then removed and tested by ELISA for TNF-αt release. Viability is analyzed using (3-4,5-dimethylthiazol-2-yl)-2,5-diphenyl-tetrazolium bromide (MTT). After 0.1 mL supernatant is collected, 0.1 mL of 0.25 mg/mL of MTT is added to remaining 0.1 mL of cells. The cells are incubated at 37° C. for about 2-4 hours, and then the optical density is measured at 490-650 nm. The IL-1β levels released in the culture medium are quantitated by ELISA. Inhibitory potency is expressed as IC₅₀

### F. VLA4 Antagonistic Activity

Compounds are tested for anti-inflammatory activity in Jurkat cell adhesion assays (J. Immunol. Methods, 164 (1993), 255-261). VCAM-1 (100 ng/well) is coated in Maxisorp microtitre module at 4° C. overnight. Non-specific blocking is carried out with 3% BSA for about 2 hours and the wells are washed with TBS (50 mM Tris, 0.15M NaCl pH 7.4, 0.1 mM CaCl₂, 0.1 mM MgCl₂, 0.1 mM MnCl₂). U937 cells are suspended in fresh medium and incubated at about 37° C. for about 2 hours before the assay. Cells are then washed in TBS solution and 180 μL of cell suspension (1×10⁶ cells/mL in TBS buffer) is added per well in VCAM-1 coated wells. 20 μL of test compound solution in 50% DMSO and 50% TBS is then added and the cells are incubated at 37° C. for about 1 hour. 3 to 5 dilutions of each test compound are tested in duplicate. After incubation, the non-adherent cells are removed by washing with TBS and the number of adhered cells quantified by lactate dehydrogenase activity estimation. The percent adhesion is calculated as compared to control.

We claim:

1. Use of a macrolide derivative for the manufacture of a medicament for treating or preventing an inflammatory disease or disorder, wherein macrolide derivative is a compound having the structure of Formula I, 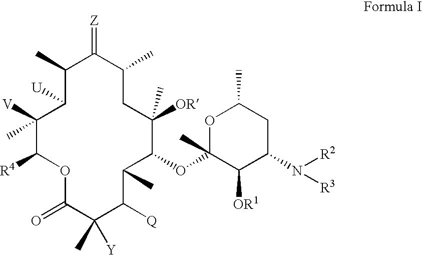
or a pharmaceutically acceptable salt, pharmaceutically acceptable solvate, polymorph, prodrug, stereoisomer, tautomeric form, N-oxide or metabolite thereof, wherein
Y can be hydrogen, halogen, cyano or alkyl;
Q can be oxo, thio, —O—(C=T)R⁵, wherein
T can be oxygen or sulfur;
R⁵ can be alkyl, alkoxy, aryl or heteroaryl;
R¹ can be hydrogen or a hydroxy protecting group;
R² and R³ can be independently alkyl, alkenyl, alkynyl, cycloalkyl, aryl, heterocycle, aralkyl, (heterocycle)alkyl or COR⁶, wherein
R⁶ can be hydrogen, alkyl, aryl or aralkyl, with the provisio that R² and R³ simultaneously are not methyl;
R′ can be hydrogen, alkyl or —(CH₂)_{q}—R⁷—, wherein
q is an integer from 1 to 4; and
R⁷ can be no atom (i.e., a bond), alkenyl or alkynyl;
Z can be O, S, NOR⁶ (wherein R⁶ is the same as defined earlier);
U and V can be independently hydrogen, hydroxy, OR¹, NH(CH₂)R⁸, wherein
R¹ is the same as defined earlier;
R⁸ can be aryl or heteroaryl; or
U and V when taken together can also form a ring of Formula A or Formula B, 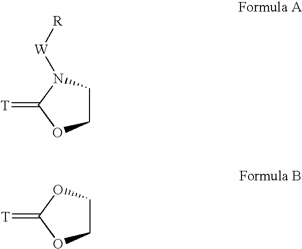
wherein
W can be alkenyl, -G(CH₂)ₘJ-, —CR⁹R¹⁰, —NR⁹— or —SO₂;
G can be no atom, —CO, —CS or —SO₂;
m can be an integer of from o to 6;
R⁹ and R¹⁰ can be independently hydrogen or alkyl;
J can be no atom, —CR⁹R¹⁰ or N(R¹²)(CH₂)ₘ;
R¹² can be hydrogen, alkyl, alkylene, alkynyl, COR¹¹ or —(CH₂)ₘ—R¹¹, wherein
R¹¹ can be alkyl, aryl or heterocycle; and
m, R⁹ and R¹⁰ are the same as defined earlier;
R can be hydrogen, aryl or heterocycle;
T is as defined earlier; and
R⁴ can be alkyl, alkenyl or alkynyl.

2. The use of claim 1, wherein the macrolide derivative has the structure of Formula Ia, 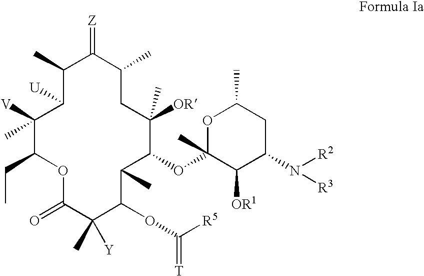
wherein Y is hydrogen, and R¹, R², R³, R′, Z, U, V, T and R⁵ are the same as defined for the compound of Formula I.

3. The use of claim 1, wherein the macrolide derivative has the structure of Formula 1b, 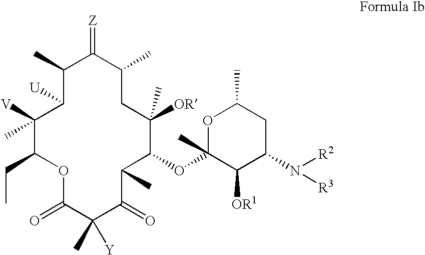
wherein Y is hydrogen, R¹, R², R³, R′, Z, U and V are the same as defined for the compound of Formula I.

4. The use of claim 1, wherein the macrolides derivate has the structure of Formula 2, 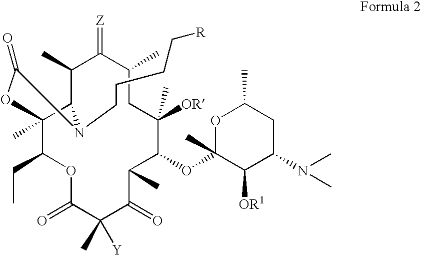
wherein Y is hydrogen or methyl; R is aryl or heterocycle; and R¹, R′, and Z are the same as defined for the compound of Formula I.

5. Use of a macrolide derivative for the manufacture of a medicament for treating or preventing an inflammatory disease or disorder, wherein macrolide derivative is, wherein macrolide derivative is selected from:
3-O-(2-Fluorophenyl)acetyl-5-O-[3′-N-desmethyl-3′-N-(4-fluoro)benzyl]desosaminyl-6-O-methyl erythronolide A,
3-O-(3-Fluorophenyl)acetyl-5-O-[3′-N-desmethyl-3′-N-cyclopropyl)desosaminyl-6-O-methyl erythronolide A,
3-O-(4-Fluorophenyl)acetyl-5-O-[3′-N-desmethyl-3′-N-(3-hydroxy)benzyl]desosaminyl-6-O-methyl erythronolide A,
3-O-(4-Fluorophenyl)acetyl-5-O-(3′-N-desmethyl-3′-N-propargyl)desosaminyl-6-O-methyl erythronolide A,
3-O-(3-Fluorophenyl)acetyl-5-O-(3′-N-desmethyl-3′-N-allyl)desosaminyl-6-O-methyl erythronolide A,
3-O-(4-Fluorophenyl)acetyl-5-O-(3′-N-desmethyl-3′-N-allyl)desosaminyl-6-O-methyl erythronolide A,
3-O-(2-Fluorophenyl)acetyl-5-O-(3′-N-desmethyl-3′-N-propargyl)desosaminyl-6-O-methyl erythronolide A,
3-O-(3-Fluorophenyl)acetyl-5-O-(3′-N-desmethyl-3′-N-propargyl)desosaminyl-6-O-methyl erythronolide A,
3-O-(2-Fluorophenyl)acetyl-5-O-(3′-N-desmethyl-3′-N-allyl)desosaminyl-6-O-methyl erythronolide A,
3-O-(4-Fluorophenyl)acetyl-5-O-(3′-N-desmethyl-3′-N-(4-nitro)benzyl)desosaminyl-6-O-methyl erythronolide A,
3-O-(4-Nitrophenyl)acetyl-5-O-(3′-N-desmethyl-3′-N-ethyl)desoaminyl-6-O-methyl erythronolide A,
3-O-(2-Pyridyl)acetyl-5-O-(3′-N-desmethyl-3′-N-cyclopropylmethyl)desosaminyl-6-O-methyl erythronolide A,
3-O-(2-Naphthyl)acetyl-5-O-(3′-N-desmethyl-3′-N-benzyl)desosaminyl-6-O-methyl erythronolide A,
3-O-(2,4-Difluorophenyl)acetyl-5-O-(3′-N-desmethyl-3′-N-benzyl) desosaminyl-6-O-methyl erythronolide A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyrrolo-(2,3-b)-pyridin-1-yl)-butyl)-imino)]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(3H)-imidazol-[4,5-b]pyridin-3-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(1H)-imidazol-[4,5-b]pyridin-1-yl)-butyl)-imino]-erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-pyrrolo-[2,3-b]pyridin-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-indol-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-quinolin-4-ylmethyl)-2-aminoethyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N-pyridine-4-ylmethyl)-2-aminoethyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-nitro)-indol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((1H)-imidazo[4,5-c]pyridin-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-2-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((3H)-imidazo-[4,5-c]pyridin-3-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-4-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-3-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-(benzotriazol-1-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-(benzoimidazol-1-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-((3H)-imidazo-[4,5-b]pyridin-3-yl)-butyl)-imino)]erythromycin A,
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A,
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-(benzotriazol-2-yl)-butyl)-imino)]erythromycin A,
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-(indol-1-yl)-butyl)-imino)]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-2-ylmethyl)-2-aminoethyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-quinolin-4-ylmethyl)-2-aminoethyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-4-ylmethyl)-2-aminoethyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A,
2α-Fluoro-5-O-(3′-N-desmethyl-3′-N-allyl)-1,12-dideoxy-3-O-decladinosyl-6-O-methyl-3-oxo-12,11-[oxycarbonyl-((N′-methyl-N′-quinoline-4-ylmethyl)-2-aminoethyl)-imino]erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyridin-3-yl-methyl-benzimidazol-1-yl)-butyl)-imino)]erythromycin A,
5-O-(3′-N-desmethyl-3″-N-ethyl)desosaminyl-11,12-dideoxy-3-O-decladinosyl-6-O-methyl-3-oxo-12,11-[oxycarbonyl-((4-(4-pyridin-3-yl-imidazol-1-yl)-butyl)-imino)]erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(imidazo[4,5-b]pyridin-1-yl)-butyl)-imino)]erythromycin A,
2α-Fluoro-5-O-(3′-N-desmethyl-3′-N-isopropyl)-11,12-dideoxy-3-O-decladinosyl-6-O-methyl-3-oxo-12,11-[oxycarbonyl-((4-((5-nitro)-indol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-fluoro)-indol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-2-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A,
2α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-pyridin-3-yl)-imidazol-1-yl)-butyl)-imino]erythromycin A,
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A,
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-(benzotriazol-1-yl)-butyl)-imino)]erythromycin A,
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-pyridin-3-yl)-imidazol-1-yl)-butyl)-imino)]erythromycin A,
2α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((N′-acetyl-N′-pyridine-3-ylmethyl)-2-aminoethyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(3H)imidazo[4,5-b]pyridin-3-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-phenyl)-imidazol-1-yl)-butyl)-imino]erythromycin A,
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-(3H)imidazo[4,5-b]pyridin-3-yl)-butyl)-imino]erythromycin A,
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A,
2α-Fluoro-5-O-(3′-N-desmethyl-3′-N-ethyl)-T 1,12-dideoxy-3-O-decladinosyl-6-O-methyl-3-oxo-12,11-[oxycarbonyl-((4-(quinolin-8-yl)-butyl)-imino)]erythromycin A,
2α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A,
2α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-pyridin-3-yl)-imidazo)-1-yl)-butyl)-imino)]erythromycin A,
11,12-dideoxy-3-O-decladinosyl-3-O-(2-pyridylacetyl)-5-O-(3′-N-desmethyl-3′-N-propargyl)-6-O-methyl-12,11-[oxycarbonyl-((4-benzo[imidazo-1-yl)-butyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A,
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyridin-2-yl-1H-imidazol-1-yl)-butyl)-imino))]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl-pyrazol-1-yl)-butyl)-imino))erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((3-pyrrolo[2,3-b]pyridin-1-yl)-propyl)-hydrazo)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((indazol-2-yl)-butyl)-imino))]erythromycin A,
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-amino-9H-purin-9-yl butylimino)]erythromycin A,
11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(isoquinolin-5-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((1-allyl-3-(9-(4-amino-butyl)-9H-purin-6-yl)-urea)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-2-yl-1H-imidazol-1-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(1H-imidazol[4,5-c]pyridin-1-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-3-yl)-1H-imidazol-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-purin-9-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-purin-9-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-isoquinolin-5-yl)-propyl)-hydrazo)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-hydrazo]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(isoquinolin-5-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-imidazol-1yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-3-yl-1H-imidazol-1yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-furan-3-yl-1H-imidazol-1yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-2-yl-1H-imidazol-1-yl)-propyl)-hydrazo)]erythromycin A,
2α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol-1-yl)propyl)-hydrazo)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-furan-2-yl-1H-imidazol-1-yl)-butyl)-imino))]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-thiophen-2-yl-1H-imidazol-1-yl)-butyl)-imino))]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-2-yl)-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-4-([1,4′]-bipyrazol-1′-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-3-yl)-propyl)-hydrazo)]erythromycin A,
2α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-thiophen-3-yl-phenoxy)-propyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(3-thiophen-3-yl-phenoxy)-propyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-thiazolyl)-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiazol-2-yl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(1H-imidazol[4,5-b]pyridin-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-tetrazol-1-yl)-imidazol-1-yl)-butylimino)]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-Imidazo[4,5-b]pyridine-2-yl)-butyl)-imino)erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(1H-benzimidazol-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Tetrazol-1-yl-imidazol-1-yl)-butyl)-imino)erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-Benzoimidazol-2-yl)-butyl)-imino)erythromycin A,
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(4-imidazol[4,5-b]pyridin-1-yl)-butylimino)]erythromycin A,
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(6-amino-9H-purin-9-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[4-(pyrazol-1-yl)-imidazol-1-yl)butylimino]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-([1,4′]-biimidazol-1′-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([1,4′]-biimidazol-1′-yl)butylimino)]erythromycin A,
11,12-dideoxy-3-O-decladinosyl-3-O-(2-methylpropanoyl)-5-O-(3′-N-desmethyl-3′-N-allyl)-6-O-methyl-12,11-[oxycarbonyl-(4-(imidazo[4,5-b]pyridin-3-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyrazol-1-yl)-imidazol-1-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiazol-2-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(4-pyrazol-1-yl)-imidazol-1-yl)-propyl)-hydrazo]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-(1H-imidazo[4,5-b]pyridine-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(oxazol-5-yl)-imidazol-1-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[3,3′]-bithiophenyl-5-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-3-yl)-tetrazol-2-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(6-chloropyridin-3-yl)-imidazol-1-yl]butylimino)]erythromycin A,
11,12-dideoxy-3-O-decladinosyl-3-O-(2-methylpropanoyl)-5-O-(3′-N-desmethyl-3′-N-allyl)-6-O-methyl-12,11-[oxycarbonyl-4-(4-(4-(furan-2-yl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-([1,4′]-bipyrazol-1-yl)-)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(2-methoxy-phenyl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-methoxy-phenyl)-1H-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-furan-2-yl-thiophen-2-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-furan-2-yl-thiophen-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A,
11,12-dideoxy-3-O-decladinosyl-3-O-(2-nitrophenyl acetyl)-5-O-(3′-N-desmethyl-3′-N-allyl)-6-O-methyl-12,11-[oxycarbonyl-(4-benzimidazol-1-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(4-pyrazol-1-yl]-imidazol-1-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(3-aminophenyl)-imidazol-1-yl)-butylimino)]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Thiophen-3-yl-pyrazol-1-yl)-butyl)-imino)erythromycin A, or
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Thiophen-2-yl-pyridin-3-yl)-butyl)-imino)erythromycin A.

6. The use of claim 1, wherein the inflammatory disease or disorder is selected from a respiratory disease, a renal disease, an autoimmune disease, a skin disease, a cardiovascular disease, Reiter's syndrome, leuko-cytoclastic vasculitis, conjunctivitis, uveoretinitis, septic shock, atherosclerosis, Crohn's disease, ulcerative colitis, colitis, rheumatoid spondylitis, osteoarthritis or septic arthritis.

7. The use of claim 1, further comprising of one or more additional agents selected from steroidal anti-inflammatory agent, non-steroidal anti-inflammatory agent, COX-2 inhibitor, macrolide immunosuppresive, p-38 MAP kinase inhibitor, local anesthetic, beta-2 agonist, 5-lipooxygenase inhibitor, phosphodiesterase inhibitor, MMP inhibitor, TNF-alpha inhibitor, caspase inhibitor, VLA4 antagonist, PAF antagonist or a mixture thereof.

8. The use of claim 5, wherein the inflammatory disease or disorder is selected from a respiratory disease, a renal disease, an autoimmune disease, a skin disease, a cardiovascular disease, Reiter's syndrome, leuko-cytoclastic vasculitis, conjunctivitis, uveoretinitis, septic shock, atherosclerosis, Crohn's disease, ulcerative colitis, colitis, rheumatoid spondylitis, osteoarthritis or septic arthritis.

9. The use of claim 5, further comprising of one or more additional agents selected from steroidal anti-inflammatory agent, non-steroidal anti-inflammatory agent, COX-2 inhibitor, macrolide immunosuppresive, p-38 MAP kinase inhibitor, local anesthetic, beta-2 agonist, 5-lipooxygenase inhibitor, phosphodiesterase inhibitor, MMP inhibitor, TNF-alpha inhibitor, caspase inhibitor, VLA4 antagonist, PAF antagonist or a mixture thereof.
